(19) United States
(12) Patent Application Publication
(10) Pub. No.: US 2017/0152219 A1
(43) Pub. Date: Jun. 1, 2017

(54) METHOD FOR THE MANUFACTURE OF UREA

(71) Applicants: Rachid Mabrouk, Munchen (DE); Klemens Wawrzinek, Grunwald (DE); Christian Voss, Geretsried (DE); Josef Schwarzhuber, Wolnzach (DE); Andreas Seliger, Munchen (DE); Benedikt Schürer, Pullach (DE); Gabriel Salazar Duarte, Munchen (DE)

(72) Inventors: Rachid Mabrouk, Munchen (DE); Klemens Wawrzinek, Grunwald (DE); Christian Voss, Geretsried (DE); Josef Schwarzhuber, Wolnzach (DE); Andreas Seliger, Munchen (DE); Benedikt Schürer, Pullach (DE); Gabriel Salazar Duarte, Munchen (DE)

(21) Appl. No.: 15/353,751

(22) Filed: Nov. 17, 2016

(30) Foreign Application Priority Data
Dec. 1, 2015 (DE) DE 102015015524.5

(51) Int. Cl. C07C 273/10; C01B 3/56; B01J 19/24; C01C 1/04; B01J 7/02; B01J 12/00; C01B 3/48; C01B 31/20

(52) U.S. Cl. CPC C07C 273/10; C01B 3/48; C01B 3/56; C01B 31/20; C01C 1/04; B01J 7/02; B01J 12/00; B01J 19/245; C01B 2210/0014; C01B 2203/025; C01B 2203/0283; C01B 2203/042; C01B 2203/068; C01B 2203/1241

(57) ABSTRACT

A method for producing urea. A methane-containing feed gas stream is reacted with oxygen by partial oxidation to form a synthesis gas stream containing hydrogen and carbon monoxide. The carbon monoxide is reacted with water in a water gas-shift reaction to form carbon dioxide and hydrogen. The synthesis gas stream is separated into a first synthesis gas substream a second synthesis gas substream. The first synthesis gas substream is subjected to pressure-swing adsorption to separate hydrogen and the second synthesis gas substream is subjected to temperature-swing adsorption to separate carbon dioxide. The separated is reacted with nitrogen to form ammonia and the ammonia is reacted with the carbon dioxide to form urea.

### The invention relates to a method for the manufacture of urea.

The synthesis of urea (H₂N—CO—NH₂) requires two important reactants, namely CO₂ and ammonia (NH₃).

The synthesis of ammonia for manufacturing the required ammonia, and the synthesis of urea can in this case be based on a steam reformation that provides the required hydrogen for the synthesis of ammonia or CO₂ for the synthesis of urea. CO₂ manufactured in this case is customarily removed by means of a scrubbing method, wherein the CO₂ is generally manufactured during the regeneration of the scrubbing medium loaded with CO₂. For this purpose, the loaded scrubbing medium is typically heated at relatively low pressure, in such a manner that there is a high energy requirement for compressing the CO₂ required in the synthesis of urea.

Ammonia and CO₂ are the main reactants for the synthesis of urea. Ammonia is usually produced by means of air-fed ATR reformers, wherein manufactured H₂ and N₂ are mixed, followed by a shift reactor and a methanization reactor for converting all of the CO to methane. CO₂ and methane are thereafter separated from the H₂—N₂ mixture. The H₂—N₂ mixture is then compressed and converted in the ammonia reactor. In an alternative layout, ammonia can be manufactured by reacting hydrogen from a steam reformer with nitrogen from an air separation unit. This layout requires, in addition to an air separation unit, a conventional hydrogen system. Hydrogen and nitrogen are mixed before being fed into the ammonia reactor and compressed. The advantage of the second layout is the low content in inerts of the ammonia synthesis gas.

The ammonia that is manufactured is then converted to urea by adding CO₂. The CO₂ manufactured in both layouts is regularly not sufficient in order to completely react the ammonia that is manufactured. Therefore, in each case CO₂ is imported from external sources, where present.

The problem addressed by the invention, against this background, is to improve a method of the type cited at the outset.

This problem is solved by a method having the features described herein.

Accordingly, it is provided according to the invention that the method for producing urea (H₂N—CO—NH₂) comprises the steps:
reacting a methane-containing and also, preferably desulphurized, feed gas stream (in particular comprising natural gas or CH₄) with oxygen by partial oxidation to form a synthesis gas stream comprising hydrogen and carbon monoxide,
reacting carbon monoxide of the synthesis gas stream in a water gas-shift reaction with water to form carbon dioxide and hydrogen,
dividing the synthesis gas stream into at least one first and one second synthesis gas substream,
separating off hydrogen from the first synthesis gas substream by means of pressure-swing adsorption, and
separating off carbon dioxide from the second synthesis gas substream by means of temperature-swing adsorption,
reacting hydrogen (H₂) separated off from the first synthesis gas substream with nitrogen (N₂) to form ammonia (NH₃), and
reacting ammonia with carbon dioxide (CO₂) separated off from the second synthesis gas substream to form urea.

For the manufacture of synthesis gas, accordingly, preferably partial oxidation (POX) is used. In this case, either a catalyst-based POX or a POX can be used which succeeds without a catalyst.

The feed gas stream preferably comprises one or more of the following components or hydrocarbons that are reacted in the synthesis gas manufacturing step to form the synthesis gas which comprises H₂ and CO; natural gas, CH₄, H₂O, CO₂.

In the partial oxidation, the preferably prepurified, in particular desulphurized (see also below) feed gas stream which comprises, e.g. natural gas or CH₄, or higher hydrocarbons such as naphtha, LPG, oil or else coal, is reacted, in particular, substoichiometrically in an exothermic process. Reaction products are primarily the materials hydrogen and carbon monoxide that form the synthesis gas and are obtained according to

CₙHₘ+(n+x)/₂O₂=(n−x+y/2)CO+(m−y)/2H₂+xCO₂+y/2 H₂O

In the partial oxidation, steam can also be added as a reactant.

In said water gas-shift reaction, to which the synthesis gas stream that is manufactured by POX is subjected, according to

CO+H₂O<−>CO₂+H₂

CO that is present in the synthesis gas is reacted with water to form carbon dioxide and hydrogen, which here is particularly advantageous, since firstly hydrogen is required for the synthesis of ammonia and CO₂ is required for the synthesis of urea.

According to an embodiment of the invention, it is further provided that the carbon dioxide that is separated off in the separation is provided at a high pressure of at least 20 bar, preferably at least 30 bar, most preferably at least 50 bar.

According to an embodiment of the invention, it is further provided that the carbon dioxide that is separated off is provided at least stoichiometrically for the reaction of ammonia to form urea, in such a manner that the ammonia (NH₃) is completely reacted to form urea.

According to an embodiment of the invention, it is further provided that in the temperature-swing adsorption for separating off the CO₂, during one cycle time, CO₂ is adsorbed from the second synthesis gas substream on an adsorber and is then desorbed, wherein the cycle time is preferably less than 360 min, preferably less than 240 min, most preferably less than 180 min.

According to an embodiment of the invention, it is further provided that for separating off the hydrogen from the first synthesis gas substream, CO₂ and CO (and also, in particular CH₄) present in the pressure-swing adsorption in the first synthesis gas substream are adsorbed on an adsorber at a first pressure, wherein, preferably, the adsorber is regenerated at a second pressure that is lower than the first pressure, wherein adsorbed CO₂ and CO (and also, in particular CH₄) are desorbed, and wherein the adsorber, for removing the desorbed CO₂ and CO (and also, in particular CH₄) is purged e.g. with hydrogen, with production of an corresponding off-gas.

According to an embodiment of the invention it is further provided that the off-gas of the pressure-swing adsorption is used as fuel, wherein, preferably the off-gas is burnt for heating the feed gas stream and/or for producing and/or superheating steam. In addition, off-gases can be burnt for producing energy, or optionally compressed once more and returned to the POX.

According to an embodiment of the invention, it is further provided that an off-gas produced in the temperature-swing adsorption and comprising H₂ and CO (and also in particular CH₄) is likewise subjected to a pressure-swing adsorption in order additionally to provide hydrogen for manufacture of the ammonia, wherein, preferably, the off-gas from the temperature-swing adsorption is subjected to said pressure-swing adsorption together with the first synthesis gas substream, and/or in that the off-gas from the temperature-swing adsorption is mixed with the off-gas (comprising CO₂ and CO and, in particular, CH₄) arising in the pressure-swing adsorption and used as fuel.

According to an embodiment of the invention, it is further provided that impurities (e.g. in the form of H₂, CH₄ and/or CO) present in the CO₂ (separated off in the temperature-swing adsorption) are removed in a purification step, preferably by means of catalytic oxidation, upstream of the reaction of the CO₂ with the ammonia to form urea.

According to an embodiment of the invention, it is further provided that the synthesis gas stream is cooled upstream and/or downstream of the water gas-shift reaction, wherein the synthesis gas stream is preferably cooled with water, with manufacture of process steam.

According to an embodiment of the invention, it is further provided that heat arising during the cooling is used for regenerating an adsorber in the temperature-swing adsorption.

According to an embodiment of the invention, it is further provided that the oxygen required for the POX is manufactured by cryogenic separation of air, wherein during each separation nitrogen is further manufactured which is reacted with the hydrogen to form ammonia.

According to an embodiment of the invention, it is further provided that the feed gas stream is conducted upstream of the partial oxidation through an adsorber unit, wherein one or more sulphur compounds that are still present in the feed gas stream are adsorbed in the adsorber unit and in this case removed from the feed gas stream.

According to an embodiment of the invention, it is further provided that the synthesis gas stream or the two synthesis gas substreams are dried downstream of the water gas-shift reaction and also upstream of the pressure-swing adsorption and also temperature-swing adsorption.

According to a further aspect of the invention, a plant for producing urea is proposed which has the features described below.

Accordingly, the plant for producing urea comprises:
a POX reactor which is configured for reacting a methane-containing and also, preferably desulphurized, feed gas stream with oxygen by partial oxidation to form a synthesis gas stream comprising hydrogen and carbon monoxide,
a water gas-shift reactor downstream of the POX reactor, which is configured for reacting carbon monoxide of the synthesis gas stream in a water gas-shift reaction with water to form carbon dioxide and hydrogen, wherein the plant is further configured to divide the synthesis gas stream arriving from the water gas-shift reactor into at least one first and one second synthesis gas substream,
a pressure-swing adsorption unit which is configured to subject the first synthesis gas substream to a pressure-swing adsorption, wherein hydrogen is separated off from the first synthesis gas substream, and
a temperature-swing adsorption unit, which is configured to subject the second synthesis gas substream to a temperature-swing adsorption, wherein carbon dioxide is separated off from the second synthesis gas substream,
an ammonia reactor which is configured for reacting hydrogen separated off from the first synthesis gas substream with nitrogen to form ammonia, and
a urea reactor which is configured for reacting ammonia with carbon dioxide separated off from the second synthesis gas substream to form urea.

The plant according to the invention is, furthermore, in further embodiments, characterized by the corresponding embodiments of the method according to the invention. In this respect, the plant is preferably configured in each case to carry out the corresponding method steps of the respective embodiment of the method according to the invention.

Further features and advantages of the invention will be explained hereinafter in the description of the figures of exemplary embodiments of the invention with reference to the figures.

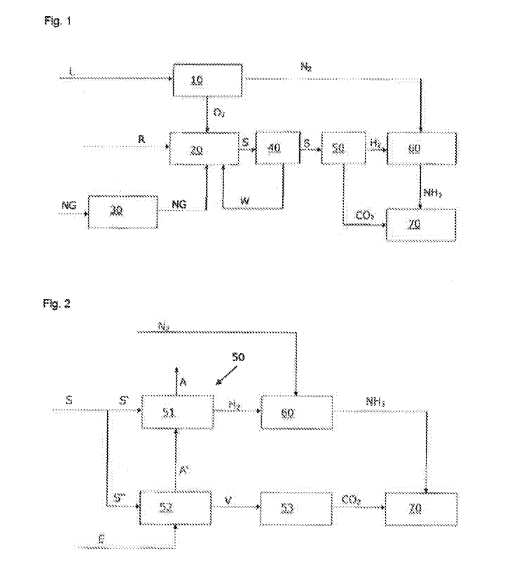

FIG. 1 shows a schematic depiction of a method according to the invention for producing urea.

FIG. 2 shows a schematic depiction of separating off CO₂ and H₂ from a synthesis gas manufactured in the method according to the invention.

FIG. 1 shows a schematic depiction of a plant and/or of a method for producing urea.

In this case, a feed gas stream NG comprising, e.g., CH₄ (e.g. in the form of natural gas), before a reaction to form synthesis gas (comprising H₂ and CO) S by partial oxidation 20 is subjected to a desulphurization 30 and then, by means of partial oxidation 20, in the presence of oxygen, and also, in particular steam W, is reacted to form a synthesis gas stream S that comprises H₂ and CO, and also further, in particular CH₄, H₂O and CO₂.

The synthesis gas stream S is hereafter subjected to a water gas-shift reaction 40 (see above) and cooled with water, wherein said steam W can be manufactured. In principle, heat arising during the cooling of the synthesis gas S can also be used for regenerating the adsorbers in the temperature-swing adsorption 51 described further below (cf. FIG. 2).

The synthesis gas stream S is in addition dried, wherein, hydrogen and carbon dioxide of the synthesis gas stream S are separated. (50), wherein the hydrogen is reacted (60) with nitrogen to form ammonia, and wherein the carbon dioxide is finally reacted with the ammonia that is manufactured to form urea.

The oxygen for the POX 20 is manufactured by cryogenic separation 10 of air L, wherein, also the nitrogen is obtained that is required for the ammonia synthesis 60.

According to FIG. 2, the hydrogen and carbon dioxide are separated off 50, preferably in such a manner that the shifted synthesis gas stream S is subdivided into a first and second synthesis gas substream S′, S″, wherein the first synthesis gas substream S′ is subjected to a pressure-swing adsorption 51, wherein hydrogen is separated off from the first synthesis gas substream S′, and wherein the second synthesis gas substream S″ is subjected to a temperature-swing adsorption 52 (see above) that is heated and/or cooled, preferably at least in part indirectly, e.g. via a heat-carrier medium that is not in direct contact with the adsorbent, wherein carbon dioxide is separated off from the second synthesis gas substream S″. The cycle times of such a temperature-swing adsorption are usually short and are in the range from 2 to 6 hours. The hydrogen that is separated off is then reacted together with the nitrogen to form ammonia 60 that in turn is reacted with the CO₂ that is separated off to form urea 70. Preferably, the CO₂ V arriving from the temperature-swing adsorption 52 is still further purified 53 upstream of the urea synthesis 70, in particular in order to remove impurities present therein such as, e.g., H₂, CH₄ and CO, methanol.

In the pressure-swing adsorption 51 for separating off the hydrogen from the first synthesis gas substream S′, CO₂ and CO and also possibly further components (such as, e.g. CH₄) that are present in the first synthesis gas substream are adsorbed on an adsorber at a first pressure, wherein, preferably the adsorber is regenerated at a second pressure which is lower than the first pressure, wherein the adsorber components are desorbed, and wherein the adsorber, for removing the desorbed components, is purged, with manufacture of an off-gas A. Preferably, a plurality, in particular two or four, adsorbers are used in the pressure-swing adsorption 51, in order that as far as possible one adsorber can always be operated in the adsorption mode in such a manner that hydrogen can be released semi-continuously.

The off-gas A from the pressure-swing adsorption 51 can be used, e.g. as fuel, wherein, e.g. the off-gas A can be burnt for heating the feed gas stream NG and/or for producing and/or superheating steam.

In the temperature-swing adsorption 52, CO₂ is adsorbed at a low first temperature on an adsorber and desorbed at a higher second temperature, for which the necessary energy E is provided. The residual gas arising in the adsorption of CO₂ and/or off-gas A′ that comprises H₂ and CO, can, together with the first synthesis gas substream S′, be run into the pressure-swing adsorption 51 or can be mixed with the off-gas A from the pressure-swing adsorption 51 and, therewith, be used together as fuel.

On account of the separation according to the invention of CO₂, said CO₂, after the separation, is advantageously present at a high pressure of preferably at least 20 bar, and so correspondingly energy can be saved for the otherwise necessary compression of the CO₂ for the purpose of urea synthesis. This is principally due to the fact that regeneration is performed during the temperature-swing adsorption by means of heating the adsorbent, and so in comparison the pressure drop occurring during regeneration in the pressure-swing adsorption is avoidable.

In addition, in the presence of the CO₂ purification 53 by catalytic oxidation, CO₂ arriving from the temperature-swing adsorption advantageously need not be cooled, since it must have a correspondingly elevated temperature for the catalytic oxidation.

The use of an appropriately designed catalytic oxidation can balance out the fluctuations in composition formed during the desorption and thus ensure a CO₂ quality as uniform as possible. The control can be adapted, in such a manner, for example, that the oxygen requirement of the catalytic oxidation is taken into account and thus an oxygen concentration in the CO₂ as constant as possible is always maintained, for example below 0.7% by volume, in particular below 0.6% by volume, or in particular <0.35% by volume. This control possibility is advantageous for the stability and energy efficiency of the subsequent urea plant. For control of the O₂ content in the CO₂, the desorption of the combustible components can be calculated in advance on account of the heating. Then, the amount of air can be set accordingly. There is also the possibility, e.g., of additionally measuring and controlling the O₂ content in the CO₂.

As a result, the invention permits the integration of known technologies such as, e.g., POX, ASU (cryogenic air separation), pressure-swing adsorption and temperature-swing adsorption, into one plant concept or method concept which can provide sufficient CO₂ for urea synthesis, and so complete reaction of the ammonia that is manufactured is possible, wherein the required CO₂ is provided at a high pressure level, and so a high-cost additional compression can he avoided.

### LIST OF REFERENCE SIGNS

| | |
|---|---|
| 10 | Air separation |
| 20 | POX |
| 30 | Desulphurization |
| 40 | Water gas-shift reaction and cooling of the synthesis gas |
| 50 | Separating off H₂ and CO₂ |
| 51 | Pressure-swing adsorption |
| 52 | Temperature-swing adsorption |
| 53 | CO₂ purification |
| 60 | Ammonia synthesis |
| 70 | Urea synthesis |
| A, | Off-gas |
| A′ | |
| E | Energy for heating |
| L | Air |
| NG | Feed gas |
| S | Synthesis gas |
| S′ | First synthesis gas substream |
| S″ | Second synthesis gas substream |
| R | Shifted synthesis gas recycle |
| V | CO₂ with impurities downstream of temperature-swing adsorption |
| W | Steam |

1. A method for producing urea, comprising the steps:
reacting a methane-containing feed gas stream with oxygen by partial oxidation to form a synthesis gas stream comprising hydrogen and carbon monoxide,
reacting the carbon monoxide of the synthesis gas stream in a water gas-shift reaction with water to form carbon dioxide and hydrogen,
dividing the synthesis gas stream into a first synthesis gas substream and a second synthesis gas substream,
subjecting the first synthesis gas substream to a pressure-swing adsorption to separate hydrogen from the first synthesis gas substream,
subjecting the second synthesis gas substream to a temperature-swing adsorption to separate carbon dioxide from the second synthesis gas substream,
reacting the hydrogen separated from the first synthesis gas substream with nitrogen to form ammonia, and
reacting the ammonia with the carbon dioxide separated from the second synthesis gas substream to form urea.

2. The method according to claim 1, wherein the carbon dioxide separated from the second synthesis gas substream is provided at a pressure of at least 10 bar.

3. The method according to claim 2, wherein the carbon dioxide is provided at a pressure of at least 20 bar.

4. The method according to claim 3, wherein the carbon dioxide is provided at a pressure of at least 50 bar.

5. The method according to claim 1, wherein the carbon dioxide separated from the second synthesis gas substream is provided stoichiometrically to the step of reaching the ammonia with the carbon dioxide to form urea, and wherein the ammonia is completely reacted to form urea.

6. The method according to claim 1, wherein, the step of subjecting the second synthesis gas substream to a temperature-swing adsorption comprises adsorbing then desorbing the carbon dioxide from the second synthesis gas substream using an adsorbent that is heated and cooled indirectly.

7. The method according to claim 6, wherein the adsorbing and desorbing of the carbon dioxide during temperature-swing adsorption comprises one cycle and wherein the time for one cycle is less than 360 minutes.

8. The method according to claim 7, wherein the time for one cycle is less than 240 minutes.

9. The method according to claim 8, wherein the time for one cycle is less than 180 minutes.

10. The method according to claim 1, wherein the step of subjecting the first synthesis gas substream to a pressure-swing adsorption comprises
adsorbing CO₂ and CO present in the first synthesis gas substream onto an adsorber at a first pressure,
regenerating the adsorber at a second pressure that is lower than the first pressure to desorb the CO₂ and CO, and
removing the desorbed CO₂ and CO by purging with production of an off-gas.

11. The method according to claim 10, wherein the off-gas is used as fuel for heating the feed gas stream or to produce or superheat steam.

12. The method according to claim 10, wherein the a temperature-swing adsorption produces an off-gas comprising H₂ and CO, wherein the off-gas is subjected to a pressure-swing adsorption to produce additional hydrogen for use in the formation of the ammonia.

13. The method according to claim 12 wherein the off-gas from the temperature-swing adsorption is subjected to the pressure-swing adsorption together with the first synthesis gas substream.

14. The method according to claim 12 wherein the off-gas from the temperature-swing adsorption is mixed with the off-gas from the pressure-swing adsorption and used as fuel.

15. The method according to claim 1, wherein the carbon dioxide separated in the temperature-swing adsorption includes impurities and wherein the impurities are removed in a purification step, upstream of the reaction to form urea.

16. The method according to claim 15, wherein the impurities are H₂, CH₄, or CO.

17. The method according to claim 15, wherein the purification step is a catalytic oxidation.

18. The method according to claim 1, wherein the synthesis gas stream is cooled upstream or downstream of the water gas-shift reaction.

19. The method according to claim 18, wherein the synthesis gas stream is cooled with water, from a manufacture of process steam.

20. The method according to claim 18, wherein heat created during the cooling of the synthesis gas stream is used to regenerate an adsorber in the temperature-swing adsorption.

21. The method according to claim 1, wherein the oxygen is manufactured by cryogenic separation of air, wherein the cryogenic separation of air also produces nitrogen, and wherein the nitrogen produced is reacted with the hydrogen to form ammonia.

22. The method according to claim 1, further comprising passing the feed gas stream through an adsorber unit upstream of the partial oxidation to adsorb sulphur compounds.

23. The method according to claim 1, wherein the synthesis gas stream is dried downstream of the water gas-shift reaction and upstream of the pressure-swing adsorption and the temperature-swing adsorption.

24. The method according to claim 22, wherein the first synthesis gas substream is dried downstream of the water gas-shift reaction and upstream of the pressure-swing adsorption and wherein the second synthesis gas substream is dried downstream of the water gas-shift reaction and upstream of the temperature-swing adsorption.

25. A plant for producing urea, comprising
a POX reactor for reacting a methane-containing feed gas stream with oxygen by partial oxidation to form a synthesis gas stream comprising hydrogen and carbon monoxide,
a water gas-shift reactor downstream of the POX reactor for reacting the carbon monoxide in a water gas-shift reaction with water to form carbon dioxide and hydrogen,
means to divide the synthesis gas stream from the water gas-shift reactor into a first synthesis gas substream and a second synthesis gas substream,
a pressure-swing adsorption unit for subjecting the first synthesis gas substream to pressure-swing adsorption, wherein hydrogen is separated from the first synthesis gas substream,
a temperature-swing adsorption unit for subjecting the second synthesis gas substream to temperature-swing adsorption, wherein carbon dioxide is separated from the second synthesis gas substream,
an ammonia reactor for reacting hydrogen from the first synthesis gas substream with nitrogen to form ammonia, and
a urea reactor for reacting ammonia with carbon dioxide from the second synthesis gas substream to form urea.
